(19) United States
(12) Patent Application Publication
(10) Pub. No.: US 2008/0207653 A1
(43) Pub. Date: Aug. 28, 2008

(54) Compositions and Method for Increasing the Efficacy of Epidermal Growth Factor Receptor Tyrosine Kinase Inhibitors

(76) Inventors: W. Jeffrey Petty, Lewisville, NC (US); Ethan Dmitrovsky, Hanover, NH (US)

Correspondence Address: LICATA & TYRRELL P.C., 66 E. MAIN STREET, MARLTON, NJ, 08053, US

(21) Appl. No.: 11/910,660

(22) PCT Filed: Apr. 7, 2006
(86) PCT No.: PCT/US2006/013196

§ 371 Date: Dec. 7, 2007

Related U.S. Application Data
(60) Provisional application No. 60/669,139, filed on Apr. 7, 2005.

(51) Int. Cl. A61K 45/00; C12N 5/00; A61P 35/00

(52) U.S. Cl. 514/266.1; 435/375

(57) ABSTRACT

Compositions and methods for increasing the efficacy of an epidermal growth factor receptor-tyrosine kinase inhibitor are provided. The drugs are modified stereospecific epidermal growth factor receptor-tyrosine kinase inhibitors that upon modification are able to accumulate at higher levels in tumor cells thereby increasing the efficacy of the inhibitor.

### INTRODUCTION

This application claims benefit of U.S. Provisional Patent Application Ser. No. 60/669,139, filed Apr. 7, 2005, the contents of which is incorporated herein by reference in its entirety.

### BACKGROUND OF THE INVENTION

Molecular targeting in cancer therapy, as opposed to conventional chemotherapy and radiation therapy, provides increased tumor specificity and reduced toxicity potential for the patient. Inhibition of epidermal growth factor receptor is one such molecular target that has been used successfully in the treatment of a variety of tumors, including tumors of the colon, head and neck, ovarian, pancreatic, and lung (Harari (2004) Endocrin. Relat. Cancer 11:689-708; Ng, et al. (2002) Mol. Cancer Ther. 1(10):777-83). The mechanism underlying the use of epidermal growth factor receptor as a target is the over expression, dysregulation or mutation of the receptor in epithelial malignancies. Examples of the therapeutic agents that have been developed include monoclonal antibodies that target the extracellular domain of the epidermal growth factor receptor (e.g., cetuximab) and inhibitors of tyrosine kinase that target the catalytic domain of epidermal growth factor receptor (e.g., gefitinib and erlotinib).

Erlotinib (CP-358774) and gefitinib (ZD-1839), known by the trade names of TARCEVA™ and IRESSA™, respectively, are both marketed as oncologic drugs in countries around the world, although the exact mechanism of action of these drugs remains to be elucidated. Gefitinib was initially approved for use in the United States as a monotherapy for treatment of locally advanced or metastatic non-small cell lung cancer after therapy with more traditional chemotherapy has failed. The doses shown to be effective in humans range from 250 to 500 mg/day (oral), doses that were well-tolerated showing little toxicity. Erlotinib has been tested clinically and approved for the treatment of patients with chemotherapy refractory non-small cell lung cancer at doses in the range of 150 mg, showing the ability to affect tumor growth with little reported toxicity (Perez-Soler (2004) Clin. Lung Cancer 6:S20-S23). In animals, erlotinib has been shown to inhibit proliferation of tumor human tumor xenografts at doses in the range of 10 mg/kg (Pollack, et al. (1999) J. Pharmacol. Exp. Ther. 291:739-748).

Erlotinib has been classified as a reversible inhibitor of human epidermal growth factor receptor tyrosine kinase. In vitro and in vivo data have indicated that this drug represses expression of cyclin D1 protein in aerodigestive tract cancers (Petty, et al. (2004) Clin. Cancer Res. 10:7547-7554). In this same study, intratumoral concentrations of erlotinib achieved in patients were shown to correlate with clinical and pathological responses. However, the exact mechanism of anti-tumor activity as well as optimal clinical doses of this agent have yet to be determined.

### SUMMARY OF THE INVENTION

The present invention is a composition containing a modified stereospecific epidermal growth factor receptor-tyrosine kinase inhibitor and a pharmaceutically acceptable carrier. In particular embodiments, the composition of the invention is used in a method for increasing the efficacy of an epidermal growth factor receptor-tyrosine kinase inhibitor. The method involves contacting a tumor cell with the composition of the present invention so that the intracellular accumulation of epidermal growth factor receptor-tyrosine kinase inhibitor is increased thereby increasing its efficacy.

### DETAILED DESCRIPTION OF THE INVENTION

Erlotinib is an anti-tumor drug used in the treatment of a variety of cancerous tumors. The exact molecular mechanism responsible for the therapeutic efficacy of the drug remains to be elucidated. It has now been found that stereoisomerization of erlotinib leads to rapid drug efflux. Therefore, modification of the chemical structure of erlotinib to prevent stereoisomerization results in an increased intracellular accumulation of erlotinib in tumor cells thereby increasing therapeutic efficacy.

Analysis of erlotinib metabolism was carried out using erlotinib-resistance cancer cells (human lung cancer A549 cells). Cells were exposed to achievable clinical doses (3 μM) of erlotinib and then lysed at various times following exposure to the drug. Relative erlotinib concentrations were determined in the A549 cell treatment media and compared to levels of erlotinib containing media incubated in the absence of cells as a control. Erlotinib concentrations were assessed using a liquid chromatography dual mass spectrometric method. Results showed that intracellular concentrations of erlotinib peaked at 4 hours, followed by a marked decrease to nearly undetectable levels by 8 hours. In the treatment media, total erlotinib concentrations did not vary significantly over the examined time course. Analytical data showed that erlotinib migrated unexpectedly as a doublet, with migration times of 1.68 minutes for the parent drug and 1.78 minutes for a previously unidentified form of the drug. Based on an analysis of chemical structure, only one stereoisomer of the parent drug could account for the 1.78 minute variant form. The stereoisomer migrating at 1.78 minutes represents the L or S configuration of this molecule, wherein the configuration is determined by performing liquid chromatographic separation of the two stereoisomers followed by nuclear magnetic resonance (NMR) analysis of each. In the absence of cells, the parent drug constituted the most abundant stereoisomer present in the media throughout the time course. In the presence of cells, however, the parent drug was converted to the 1.78 minute stereoisomer during the course of the experiment, with some conversion to the form seen at 4 hours and complete conversion to the 1.78 minute form seen at 8 hours, coinciding with the maximal decline in intracellular erlotinib concentrations.

The results showed that parent stereoisomer accumulates in cells but that stereoisomerization led to rapid drug efflux or lack of influx in the erlotinib-resistant cells. Therefore, modification of the chemical structure of erlotinib in order to prevent stereoisomerization can effectively increase drug accumulation intracellularly leading to improved therapeutic efficacy based on the correlation that exists between efficacy and intracellular drug accumulation.

Accordingly, the present invention is composition containing a modified stereospecific epidermal growth factor receptor (EGFR)-tyrosine kinase inhibitor administered in a pharmaceutically acceptable carrier. As used herein, a stereospecific EGFR-tyrosine kinase inhibitor is intended to mean a specific efficacious stereoisomer of, e.g., erbstatin analog, 2,5-dihydroxymethylcinnamate; erlotinib, N-(3-ethynylphenyl)-6,7-bis(2-methoxyethoxy)quinazolin-4-amine; gefitinib, 4-(3-chloro-4-fluoroanilino)-7-methoxy-6-(3-morpholino propoxy) quinazoline; PD 153035, 4-[(3-bromophenyl)amino]-6,7-dimethoxyquinazoline hydrochloride; tyrphostin AG 490, N-benzyl-3,4-dihydroxy-benzylidenecyanoacetamide; tyrphostin AG 825, (E)-3-[3-[2-benzothiazolythio)methyl]-4-hydroxy-5-methoxyphenyl]-2-cyano-2-propenamide and cetuximab. In particular embodiments, the modified stereospecific EGFR-tyrosine kinase inhibitor is erlotinib.

As will be appreciated by the skilled artisan, the stereospecific EGFR-tyrosine kinase inhibitor of the instant invention can be modified in accordance with various art-established medicinal chemistry methods to prevent stereoisomerization into a non-efficacious or less efficacious stereoisomer. For example, the stereospecific inhibitor can be conjugated to an inactive moiety which maintains the stereospecificity of the inhibitor without affecting the interaction with the (EGFR)-tyrosine kinase. Contemplated modifications include alterations in the chemical backbone to increase steric hindrance. Additional modifications include liposomal encapsulation to protect the drug in a specific steric conformation until it reaches the desired target cells. By way of illustration, selectivity and toxicity of antitumor drug chlorambucil was improved by conjugation with albumin via carboxylic hydrazone bonds (Kratz, et al. (1999) Archiv der Pharmazie 331:47-53). Likewise, it is contemplated that a stereospecific antibody, which does not block EGFR-tyrosine kinase inhibitor activity, can be conjugated to or co-administered with the inhibitors, thereby maintaining the preferred isomeric structure of the inhibitor. The generation of such stereospecific antibodies is well-established in the art. See, e.g., Chikhi-Chorfi, et al. ((2001) Chirality 13:187-92), which teach the generation of selective antibodies to (R)-methadone and its racemate. Other suitable conjugates and chemical modifications for maintaining stereospecificity are well-known to the skilled artisan.

The effectiveness of any particular conjugate or modification for maintaining the preferred isomeric structure of the EGFR-tyrosine kinase inhibitor can be monitored using the cell-based assay disclosed herein, or a similar assay suitable for any particular parent compound. For example, to monitor the stereoisomer accumulation of gefitinib, a gefitinib-resistant cell line can be used.

The compositions of the instant invention can be prepared by methods and contain pharmaceutically acceptable carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A carrier, pharmaceutically acceptable carrier, or vehicle, such as a liquid or solid filler, diluent, excipient, or solvent encapsulating material, is involved in carrying or transporting the subject compound from one organ, or portion of the body, to another organ, or portion of the body. Each carrier must be acceptable in the sense of being compatible with the other ingredients of the formulation and not injurious to the patient.

Examples of materials which can serve as carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Compositions of the instant invention can be administered via any route including, but not limited to, oral, rectal, topical, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intrathecal and inhalation administration, as well as direct organ injection (e.g., into the pancreas). The most suitable route in any given case will depend on the nature and severity of the cancer being treated and on the nature of the particular modified stereospecific inhibitor being used.

For injection, the carrier will typically be a liquid, such as sterile pyrogen-free water, pyrogen-free phosphate-buffered saline solution, bacteriostatic water, or Cremophor® (BASF, Parsippany, N.J.). For other methods of administration, the carrier can be either solid or liquid.

For oral therapeutic administration, the modified stereospecific inhibitor can be combined with one or more carriers and used in the form of ingestible tablets, buccal tablets, troches, capsules, elixirs, suspensions, syrups, wafers, chewing gums, foods and the like. Such compositions should contain at least 0.1% of active compound. The percentage of the modified stereospecific inhibitor in the composition can, of course, be varied and can conveniently be between about 0.1 to about 100% of the weight of a given unit dosage form. The amount of active inhibitor in such compositions is such that an effective dosage level will be obtained.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring. The above listing is merely representative and one skilled in the art could envision other binders, excipients, sweetening agents and the like. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like.

A syrup or elixir can contain the active agent, sucrose or fructose as a sweetening agent, methyl and propylparabens as preservatives, a dye and flavoring such as cherry or orange flavor. Of course, any material used in preparing any unit dosage form should be substantially non-toxic in the amounts employed. In addition, the active inhibitors can be incorporated into sustained-release compositions and devices including, but not limited to, those relying on osmotic pressures to obtain a desired release profile.

Compositions of the present invention suitable for parenteral administration contain sterile aqueous and non-aqueous injection solutions of the modified stereospecific inhibitor, which preparations are generally isotonic with the blood of the intended recipient. These preparations can contain anti-oxidants, buffers, bacteriostats and solutes which render the composition isotonic with the blood of the intended recipient. Aqueous and non-aqueous sterile suspensions can include suspending agents and thickening agents. The compositions can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Compositions suitable for topical application to the skin can take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Compositions suitable for transdermal administration can be presented as discrete patches adapted to remain in intimate contact with the epidermis of the recipient for a prolonged period of time. Compositions suitable for transdermal administration can also be delivered by iontophoresis (see, for example, Pharmaceutical Research 3 (6):318 (1986)) and typically take the form of an optionally buffered aqueous solution of the compound. Suitable Compositions contain citrate or bis-tris buffer (pH 6) or ethanol/water and the stereospecific inhibitor.

A modified stereospecific inhibitor can alternatively be formulated for nasal administration or otherwise administered to the lungs of a subject by any suitable means. In particular embodiments, the compound is administered by an aerosol suspension of respirable particles containing the modified stereospecific inhibitor, which the subject inhales. The respirable particles can be liquid or solid. The term aerosol includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Crit. Rev. Therap. Drug Carrier Syst. 6:273-313; and Raeburn, et al. (1992) J. Pharmacol. Toxicol. Methods 27:143-159. Aerosols of liquid particles containing the modified stereospecific inhibitor can be produced by any suitable means, such as with a pressure-driven aerosol nebulizer or an ultrasonic nebulizer, as is known to those of skill in the art. See, e.g., U.S. Pat. No. 4,501,729. Aerosols of solid particles containing the modified stereospecific inhibitor can likewise be produced with any solid particulate medicament aerosol generator, by techniques known in the pharmaceutical art.

Alternatively, one can administer the composition containing modified stereospecific inhibitor in a local rather than systemic manner, for example, in a depot or sustained-release formulation.

Further, the present invention provides liposomal formulations containing the modified stereospecific inhibitor disclosed herein. The technology for forming liposomal suspensions is well-known in the art. When the modified stereospecific inhibitor is an aqueous-soluble salt, conventional liposome technology can be employed. In such an instance, due to the water solubility of the modified stereospecific inhibitor, the inhibitor is substantially entrained within the hydrophilic center or core of the liposomes. The lipid layer employed can be of any conventional composition and can either contain cholesterol or can be cholesterol-free. When the modified stereospecific inhibitor is water-insoluble, again employing conventional liposome formation technology, the inhibitor can be substantially entrained within the hydrophobic lipid bilayer which forms the structure of the liposome. In either instance, the liposomes which are produced can be reduced in size, as through the use of standard sonication and homogenization techniques.

A liposomal formulation containing a modified stereospecific inhibitor disclosed herein, can be lyophilized to produce a lyophilizate which can be reconstituted with a carrier, such as water, to regenerate a liposomal suspension.

The composition of the present invention can be administered to an animal, including humans, companion animals or livestock in the treatment of a cancerous tumor associated with EGFR-tyrosine kinase. Examples of such cancers include, but are not limited to, colon, head and neck, ovarian, pancreatic, breast, and lung (e.g., non-small cell lung cancer).

Given that stereoisomerization of a parent EGFR-tyrosine kinase inhibitor leads to rapid drug efflux or lack of influx in resistant cells, the present invention is a also a method for increasing the therapeutic efficacy of an EGFR-tyrosine kinase inhibitor by increasing intracellular accumulation of the EGFR-tyrosine kinase inhibitor. The method involves contacting a tumor cell with a composition containing a modified stereospecific EGFR-tyrosine kinase inhibitor, wherein contact of the tumor cell with the composition results in an increased accumulation of the modified stereospecific inhibitor as compared to the tumor cell contacted with a composition containing a non-modified inhibitor. The amount of modified stereospecific inhibitor in the composition applied to the tumor cell can be at doses established in the art. It is contemplated that because the parent inhibitor is modified to prevent stereoisomerization which may lead to increased efflux or decrease inhibitory activity, art-established dosages will be suitable and be more effective as a result of the modification. In particular embodiments, the modified stereospecific inhibitor will cause a 10%, 20%, 30%, 40%, 50%, 60%, 70%, 80%, 90% or 100% increase in the accumulation of modified stereospecific inhibitor in the tumor cell as compared to a similar tumor cell contacted with a non-modified inhibitor (i.e., the parent inhibitor compound). Such accumulation can be monitored based upon methods disclosed herein or other suitable methods for monitoring accumulation of a stereoisomer (e.g., immunoassays using stereoselective antibodies).

Other methods for increasing intracellular accumulation of an EGFR-tyrosine kinase inhibitor are also contemplated by the present invention. Such methods include inhibition of drug efflux or P450 metabolism or other pathways. Such methods would increase intracellular accumulation of an EGFR-tyrosine kinase inhibitor such as erlotinib, and its related compounds, and ultimately lead to improvements in anti-neoplastic efficacy of EGFR-tyrosine kinase inhibitors. As will be appreciated by one of skill in the art, such methods will be useful for improving therapeutic efficacy of often difficult-to-treat cancers.

The following non-limiting examples are provided to further illustrate the nature of the present invention.

### EXAMPLE 1

### Cell Culture

Culture of A549 human lung cancer cells were carried out in Ham's F12 media with L-glutamine containing 10% fetal bovine serum, amphoterecin B, penicillin, and streptomycin. Cells were plated at a density of 300,000 cells per well in a 6 well plate. Cells were allowed to adhere overnight, media was aspirated and 3 mL of treatment media containing 3 μM erlotinib was added to each well. At the same time, 3 mL of the same media was added to wells that contained no cells as a control. One hundred μL of the media was removed from the wells immediately prior to harvesting the cells at various time points. The remaining media was removed and 250 μL of distilled deionized water was added to each well. Cells were also disrupted with a rubber policeman. Each media or cell extract sample was brought to a 1 mL volume with methanol and water (final concentration 50% methanol). Fifty μL of 0.4 M ZnSO₄ was added to each sample. Samples were vortexed for 10 seconds and centrifuged at 13,000 RCF for 5 minutes. The supernatants from each were subjected to LC-MS-MS chromatographic analysis using a Waters® Model 2795 system equipped with a Waters® X-Terra® MS column. The mobile phase was composed of 70% methanol containing 0.1% formic acid at a flow rate of 0.15 mL per minute. Dual mass spectroscopy was performed with 394 for the initial mass and 278 for the daughter ion (after collision using argon gas). Dual mass spectroscopic analysis of midazolam was used as an internal standard and was performed at the same time using 326 as the mass for the parent and 291 for the daughter ion. Data was collected and analyzed using Masslynx™ version 3.4 software.

What is claimed:

1. A composition comprising a modified stereospecific epidermal growth factor receptor-tyrosine kinase inhibitor and a pharmaceutically acceptable carrier.

2. A method for increasing the efficacy of an epidermal growth factor receptor-tyrosine kinase inhibitor comprising contacting a tumor cell with the composition of claim 1 so that the intracellular accumulation of epidermal growth factor receptor-tyrosine kinase inhibitor is increased thereby increasing the efficacy of the epidermal growth factor receptor-tyrosine kinase inhibitor.
